(19) United States
(12) Patent Application Publication
(10) Pub. No.: US 2005/0079218 A1
(43) Pub. Date: Apr. 14, 2005

(54) Matrices for the stabilizing and controlled release of problematic substances

(76) Inventors: Achim Gopferich, Sinzing (DE); Angelika Maschke, Regensburg (DE)

Correspondence Address: SALTER & MICHAELSON;THE HERITAGE BUILDING, 321 SOUTH MAIN STREET, PROVIDENCE, RI, 029037128, US

(21) Appl. No.: 10/498,722

(22) PCT Filed: Dec. 12, 2002
(86) PCT No.: PCT/EP02/14172

(30) Foreign Application Priority Data
Dec. 12, 2001 (DE) 101 61 078.5

(51) Int. Cl. A61K 9/22

(52) U.S. Cl. 424/468

(57) ABSTRACT

The invention concerns a carrier system for stabilizing and controlled release of active substances, in particular problematic pharmaceutical substances in a biological environment. Said carrier system comprises a lipid matrix having water solubility corresponding to one part of the lipid matrix for 30 parts of water, and at least a release controlling substance, said substance being insoluble in the lipid matrix.

The present invention relates to carrier systems for problematic active substances such as sensitive medicaments, which both ensure the stability of the active substances and at the same time allow release over a period of days, weeks or months.

Problematic active substances such as sensitive medicaments, for example proteins, peptides and even some cytostatics, are characterised by very short half-lives in biological media that are sometimes in the region of only a few minutes. Only a very short duration of action can anticipated when sensitive medicaments such as this are introduced into the body. As a result, a great many therapeutically interesting medicaments never quite reach the stage of processing to produce effective and potent drugs. As early as the beginning of the 70s, these substances began to be protected from accelerated degradation by slow release from a repository; at the same time prolonged release from a matrix began to be used.

Polymers in particular have been investigated very intensively for their suitability as a matrix for the parenteral administration of sensitive medicaments. A great many compounds were simultaneously synthesised for this purpose. Alongside synthetic materials such as, for example, poly(α-hydroxy esters) or polyanhydrides, natural materials such as e.g. collagen or alginate are also increasingly being used.

The feature common to all these substances is that the active substances to be protected during parenteral administration are released from a repository in a controlled manner. Due to these developments, a number of therapeutically relevant substances such as, for example, LHRH agonists, somatotropin or human growth hormone have been successfully marketed as pharmaceutical forms. This is, however, only a relatively small proportion of the medicaments for which this technology would be suitable.

The reasons for this reside in the problems which arise when polymers, in particular ones which are biodegradable, are combined with active substances in protein medicaments. Thus, in the commonly used poly(α-hydroxy esters) incompatibility reactions arise between the polymer and the protein or peptide medicaments. One might cite in this regard the low pH which is possible within polymer matrices of this type. These pH values have been reported to be in the region of 2, and they therefore have a detrimental effect on some protein medicaments which leads to a loss of biological activity.

It has furthermore been established that, during degradation of the polymer matrix, in the course of which the corresponding oligomers and monomers are released, an accumulation of these degradation products occurs.

On account of this accumulation of degradation products, the osmotic pressure within the matrix is increased to 2 to 3 times the osmotic pressure of the serum or of an isotonic saline solution.

In the specific case of poly(α-hydroxy esters), such as e.g. poly(lactic acid) (PLA) or poly(lactic acid-co-glycolic acid) (PLGA) and polyanhydrides, it has also been found that the monomers occurring in the course of degradation of the polymer are covalently bound to functional groups of the active substances, for example amino groups. The reaction with polymers and/or polymeric degradation products, non-physiological pH and osmotic pressure values, can influence, and to a considerable degree impair, the efficacy and tolerability of protein and peptide medicaments.

The natural polymers include a number of substances which do not exhibit these problems, e.g. collagen, gelatins or alginate as hydrogel-forming polymers, which in the presence of water form systems with increased viscosity and in some cases elasticity. The same is true of synthetic hydrogel-forming polymers, such as cellulose ethers, polyvinyl alcohol and derivatives of polyacrylic acid. Such materials do have other disadvantages, however.

In many cases, an undesirably rapid release of the incorporated active substances occurs. As a result, it is almost impossible to release substances over longer periods of time, for example weeks or months. To achieve this, the polymer chains have to be cross-linked. Cross-linking substances such as e.g. aldehydes have been used for this purpose. It is not possible to use substances of this type for protein and peptide medicaments, however.

When alginate is used, undesirable ionic interactions with the alginate chains or the bivalent metal ions used for cross-linking, e.g. calcium ions, may also occur.

Once cross-linking has been performed, charging is very complicated, however. Charging is usually carried out by incubating the polymer in a peptide or protein solution. However, the time needed for this corresponds roughly to the release time, and is therefore uneconomically long.

Many of the materials mentioned above also have the disadvantage that they undergo considerable expansion; this greatly impedes and limits their use in many tissues, such as the brain, which is especially sensitive to pressure.

It has been known to use lipids for the release of medicaments. However, lipids in general have only a limited capacity for the controlled release of active substances. Trials were therefore conducted with control of the release of the active substances via the composition of the lipid matrices.

Thus U.S. Pat. No. 4,452,775 describes the use of different qualities of cholesterol. U.S. Pat. No. 4,610,868 describes the use of surface-active substances which cause the matrices to “dissolve”, thereby controlling their release from the matrices. In these systems, release control is achieved by working lipids up with one another and with substances that are miscible with one another or at least partially soluble in one another. This can be recognised for example from the fact that in each case, after working up these substances, no separation into the individual components occurs. If spatial separation of the individual components is a necessary consequence of the type of processing, for example the compression of powders, the substances are nevertheless at least partially soluble in one another and are in addition characterised by the fact that, in water, they are “sparingly soluble” (less than 1 part of the substance dissolves in 30 to 100 parts of water) to “practically insoluble” (less than 1 part of the substance dissolves in 10,000 parts of water).

The use described in the literature to date, of substances that are partially soluble in lipids to modify the release from lipid matrices, is associated with a number of serious disadvantages. To give an example, a change in the properties of the lipid occurs; this may be a change in its physical characteristics, such as a shift in the melting point. Furthermore, this kind of release modification offers only limited scope to vary the release without fundamentally altering the system.

U.S. Pat. No. 4,610,868 describes, for example, the use of surfactants which may, however, have a disadvantageous influence on the structure of the proteins, e.g. on their tertiary structure, and may even cause unfolding.

The disadvantage with many of the known systems is their high content of the medicament. Thus U.S. Pat. No. 5,801,141 describes the production of a lipid matrix for the parenteral administration of growth factors, which is 20 to 80% charged with the active substance. The ratio of active substance to lipid in such cases is 0.7 and above.

U.S. Pat. No. 4,985,404 describes systems consisting of oils having a polypeptide content of at least 10%. Such a high charge is unnecessary for many applications and is not economical. Furthermore, if the active-substance charge is very high, the desired controlled release is no longer guaranteed.

The above carrier systems for problematic medicaments thus all have serious disadvantages which may lead on the one hand to impairment of the stability of the active substances and on the other to a release which is difficult to control, especially when the aim is release over weeks and months

The aim of the invention was to provide a biologically safe biodegradable and bioerodable carrier system for the controlled administration of problematic active substances, in particular problematic medicaments. The aim of the invention was in particular to provide a carrier system of this type for parenteral administration.

According to the invention, an alternative was in particular to be found to the known biodegradable polymers for the release of protein and peptide medicaments which enables a controlled release of active substances of this type in vivo over days, weeks and months; in addition, the matrix material itself was to have adequate stability in vivo and at the same time create an environment for the guest molecules which does not impair their stability. With the carrier system, it was also to be possible to provide release systems for medicaments with which administration is also possible by injection.

This aim is achieved by means of a carrier system for active substances, in particular medicaments, containing at least one lipid matrix which has a solubility in water of one part lipid matrix in at least 30 or more parts of water and at least one release-controlling substance which is insoluble in the lipid matrix.

The present invention also relates to a composition containing the carrier system according to the invention and at least one active substance.

The invention relates in particular to carrier systems of this type and to pharmaceutical compositions for problematic active substances such as problematic medicaments.

With the carrier system according to the invention, any active substances may be introduced into biological environments such as those present in the bodies of humans and animals, and released there in a controlled manner.

Controlled release means that the active substance is released continuously or discontinuously into the environment over a desired time period.

According to the invention, the carrier system is preferably solid at body temperature and does not melt in the body.

Problematic active substances such as problematic medicaments within the scope of the invention are active substances or medicaments which have only very short half-lives in biological media and/or have only a narrow range of local tolerability and/or a low therapeutic index. In particular they are active substances having a half-life of less than one hour, in particular of only a few minutes. They may be active substances which degrade easily even at room temperature or under environmental conditions, and/or which are subject to rapid local enzymatic degradation in vivo.

A low therapeutic index means that the active substances must be administered in very precise dosages in order to avoid toxic effects, as the gap between therapeutic and toxic doses is small.

Examples of problematic medicaments of this type are proteins, peptides, antisense oligonucleotides, bisphosphonates, and even some cytostatics. Thus the cytostatic carmustine (BCNU) in plasma has a half-life of only approximately 20 minutes. Other examples are the enzyme hyaluronidase and D-arginyl-L-arginyl-L-prolylglycyl-3-(2-thienyl)-L-alanyl-L-seryl-(3R)-1,2,3-tetrahydro-3-isoquinolinocarbonyl-(2S,3aS,7aS)-octahydro-1H-indo-1-2-carbonyl-L-arginine, also called “HOE 140”, insulin, growth factors and cytokines, e.g. from the BMP family or the IGF family, and substances such as FGF, EGF, PDGF, NGF, BDNF and GDNF, erythropoietin, somatostatin and atrial natriuretic peptide.

Further examples are doxorubicin, 4′-epi-doxorubicin, 4 or 4′-desoxydoxorubicin or a compound preferably from the group etoposide, N-bis(2-chloroethyl)-4-hydroxyaniline, 4-hydroxycyclophosphamide, vindesine, vinblastin, vincristine, terfenadine, fexofenadine, terbutaline, fenoterol, salbutamol, muscarine, oxyphenbutazone, salicylic acid, p-aminosalicylic acid, 5-fluorouracil, methotrexate, diclofenac, flufenaminic acid, 4-methylarninophenazone, theophylline, nifedipine, mitomycin C, mitoxantron, camptothecin and camptothecin derivatives, m-AMSA, taxol, docetaxel, nocodaxol, colchicine, cyclophosphamide, rachelmycine, cisplatin, melphalane, bleomycin, nitrogen-mustard gas, phosphoramide mustard gas, verrucarin A, neocarcinostatin, calicheamicin, dynemicin, esperamicin A, quercetin, genistein, erbstatin, tyrphostine, rohitukin derivative, retinolic acid, butyric acid, phorbol ester, dimethylsulfoxide, aclacinomycin, progesterone, busereline, tamoxifen, mifepristone, onapristone, N-(4-aminobutyl)-5-chloro-2-naphthalenesulfonamide, pyridinyloxazol-2-one, quinolyloxazol-2-one, isoquinolyloxazol-2-one, staurosporin, ethanolamine, verapamil, forskoline, 1,9-dideoxyforskoline, quinine, quinidine, reserpine, ramipril, teicoplanin, risedronate, irinotecan, glatriamer acetate, riluzol, glimeperide, 18-O-(3,5-dimethoxy-4-hydroxybenzoyl) reserpate, lonidamine, buthionine sulfoximine, diethyldithiocarbamate, cyclosporin A, rapamycin, azathioprin, chlorambucil, hydroxycrotonic acid amide derivative-2, leflunomide, 15-deoxyspergualine, FK 506, ibuprofen, indomethacin, aspirin, sulfasalazine, penicillamine, chloroquine, dexamethasone, prednisolone, mefonamidic acid, paracetamol, 4-aminophenazone, muskosin, orciprenaline, isoprenaniline, amiloride and p-nitrophenylguanidine benzoate.

It will be understood that the present invention is not limited to the aforementioned examples, but is generally applicable to active substances, in particular active substances the administration of which is associated with the aforementioned problems.

The carrier system according to invention has a lipid matrix composed of one or more lipids, the lipid matrix being sparingly soluble to practically insoluble in water. This means that one part of the lipid matrix is soluble in 30 parts or more of water.

For the definition of solubility in water, according to the invention reference is made to the relevant information in the European Pharmacopoeia, vol. 1, general section, 4^{th} edition, Grundwerk 2002, Deutscher Apothekerverlag Stuttgart, Govi-Verlag-Pharmazeutischer Verlag GmbH Eschbom, page 6. According to the classification given therein, sparingly soluble to practically insoluble means that one part of the lipid matrix is soluble in 30 parts or more of water, in particular in 30 parts to 10,000 parts of water.

The carrier systems according to invention preferably exhibit no pronounced expansion and thus no marked weight increase in water. Thus the weight increase in water due to swelling is preferably below 20%, in particular below 10%, and especially preferably below 5%.

Because swelling is slight, the carrier systems according to the invention may also be used in pressure-sensitive regions of the body.

Examples of lipids are monoglycerides, diglycerides and triglycerides, the esters of glycerol, and fatty acids. The glycerol may be esterified with the same fatty acid or with different fatty acids.

Essentially any fatty acids may be used.

The length of the carbon chain of the fatty acid is determined by the type of carrier system required. Short-chain fatty acids generally lead to free-flowing to liquid systems and, correspondingly, longer-chain ones to solid systems. For parenteral administration especially solid carrier systems are desirable, and correspondingly longer-chain fatty acids may be used. Suitable examples are fatty acids having 12 or more carbon atoms.

The fatty acids should furthermore exhibit adequate stability, in view of which saturated fatty acids are preferred.

Examples of suitable glycerides are glyceryl trilaurate C12, glyceryl trimyristate with C14, glyceryl tripalmitate with C16, glyceryl tristearate C18 etc.

Other examples of suitable lipids are substances which according to the literature are known to the person with ordinary skill in the art overall as lipids, such as wax alcohols, fatty acids, ceramides, cholesterol, sphingolipids, phospholipids or lecithin.

Waxes may also be used. Examples are vegetable and animal waxes such as carnauba wax, beeswax, shellac wax or spermaceti. Synthetic waxes may also be used, the basic chemical structure of which corresponds to that of the natural waxes, e.g. synthetic spermaceti.

Lipids as defined by this invention also include synthetic or semi-synthetic substances which are biologically safe and have the requisite low solubility in water according to the invention. The lipids used according to the invention may also be corresponding substances which are known from the metabolism of fat. Examples of lipids of this type are described in U.S. Pat. No. 5,785,976, U.S. Pat. No. 6,120,789 and U.S. Pat. No. 5,888,533, to which reference is expressly made in this regard.

The advantage of the lipids used according to the invention is that, on account of their low solubility in water, they form matrices which are stable for lengthy periods in water or an aqueous biological medium, or represent systems which partially erode with the formation of colloid-disperse systems.

The lipids according to invention are thus stable in water, but are broken down in a biological environment. The degradation in a biological environment may, for example, be enzymatic, or it may take place due to natural metabolic processes.

Surprisingly it has been shown that problematic active substances such as problematic medicaments are stabilised by the lipid matrices used according to the invention.

Thus the lipids used according to the invention, in contrast with biodegradable polymers, characterised in that they do not form covalent bonds with the active substances used. Moreover, the erosion of the lipid matrix according to the invention is not associated with an increase in the osmotic pressure or a decrease in the biological pH in or around the carrier.

Surprisingly, it has been shown that although lipids such as those from the glyceride series have hydrolysable ester bonds leading, in the case of the poly(α-hydroxy esters), to the acylation of amino groups, unlike polymers they do not give rise to a significant increase in the osmotic pressure, a lowering of the pH, acylation of proteins or pronounced swelling.

The carrier system according to invention furthermore contains a release-controlling substance which governs and controls the release kinetics of the active ingredient. By the addition of the at least one release-controlling substance, even in the case of lipids it is possible to achieve a desirable release profile over a period of days, weeks or months.

The release-controlling substance used according to the invention is a substance which is not soluble in the lipid matrix or is not miscible therewith. Surprisingly, substances which are not soluble or miscible with the lipids were found to be especially advantageous for controlling the release kinetics of active substances in lipids and therefore for modifying their release. According to the invention, substances with a solubility in the lipid matrix of one part of substance to at least 10,000 parts of lipid are used for this purpose.

The solubility of the release-controlling substance in the lipid matrix is similarly classified in accordance with the solubility data given in the commentary to the German Pharmacopoeia, 7^{th} edition 1968, Wissenschaftliche Verlagsgesellschaft mbH Stuttgart, Govi-Verlag GmbH Frankfurt, pages 8 and 9.

The release-controlling substances used according to the invention have a solubility in water of 1 part of substance to 1 to 10 parts of water, in particular 10 to 30 parts of water, and may thus be classified as soluble to freely soluble.

The release-controlling substances used according to the invention may be low-molecular freely to very freely water-soluble substances selected from among electrolytes, monosaccharides and disaccharides, and amino acids such as e.g. glycine. Examples of suitable electrolytes are combinations of cations and anions from the Hofmeister series, such as those described in “Peptide and protein delivery”, V. H. L. Lee (ed.), Marcel Dekker 1991, page 179. Preferred examples of cations are Mg²⁺, Li²⁺, Na⁺, K⁺, NH⁴⁺, Zn²⁺ and Ca²⁺, and anions (SO₄)²⁻, (HPO₄)²⁻, CH₃COO⁻, Cl⁻, (NO₃)⁻ and I⁻.

Suitable examples of monosaccharides are glucose, fructose, galactose and mannose. Suitable examples of disaccharides area trehalose, gentiobiose, maltose, saccharose and lactose.

Sugar alcohols may also be used. Examples are sorbitol and mannitol.

Polymers have proved especially suitable. Suitable polymers have the capacity to swell in water, and thus to undergo a volume increase. By way of example one may cite polymers from the class of the hydrogel-forming polymers, the polysaccharides, proteins and peptides, the polyethers and the polyesters. The following substances may be used inter alia: gelatins, traganth, methyl cellulose, polyvinylpyrrolidone, agar, alginates and their salts, gum arabic, methyl cellulose, hydroxyethyl cellulose, carboxymethylcellulose, hydroxymethyl cellulose, methylhydroxypropyl cellulose, hydroxypropyl cellulose, chitosan, scleroglucans, polyacrylates or methacrylates and their copolymers, polyacrylamides, pectin, starch and starch derivatives, polyvinyl alcohol, polyethylene oxide, dextran and heparin.

Polyethylene glycol, hydrogel-forming polymers such as gelatins, alginate and collagen are especially preferred.

Non-animal polymers are also preferably used according to the invention for the release-controlling substance.

Examples of non-animal polymers are cellulose derivatives such as cellulose ethers, inter alia methyl cellulose, hydroxypropyl methyl cellulose and carboxymethyl cellulose, polyvinylpyrrolidone, and polyvinyl alcohol.

The content of the release-controlling substance in the carrier system according to the invention is usually within a range from 0.001 to 30% (m/m), with reference to the lipid matrix fraction; preferably within a range from 0.01 to 20% (m/m), and especially preferably 0.1 to 10% (m/m).

The content of release-controlling substance in the carrier system according to the invention represents a simple means whereby the rate of release of the carrier system for an active ingredient may be controlled. The determination of a suitable content as a function of the type of lipid matrix and active substance used is a routine practice which is within the capability of the person with ordinary skill in the art.

In order to adjust the desired small weight increase and thus swelling of the carrier system according to the invention in water, when using polymers which expand greatly it may be necessary to use the latter only in a small proportion or to use the polymers in a form which is not subject to pronounced swelling.

To this end, the polymers may, for example, in the first instance be swollen up in water, and the fully swollen gels obtained then freeze-dried. The polymers may then be used after appropriate comminution, without further swelling taking place.

The ratio of active-substance content to lipid content according to the invention is below 0.7 and especially 0.5 (expressed as a weight ratio) in order to ensure adequate stabilisation.

The carrier system preferably contains 30 wt. % or less, especially 15 wt. % or less, and especially preferably less than 10 wt. % of the active substance, and especially 5 wt. % and less (with reference to the carrier system).

Particularly in the case of low active-substance contents of less than 10 wt. %, the release occurs by diffusion processes, during which the desired release profile may be especially advantageously established.

The erosion duration of the carrier system according to the invention may, if required, also be shortened by addition of surface-active compounds. Examples of surface-active compounds of this type are cholesterol, glycerophospholipids and sphingophospholipids.

By this means, for example by adding phospholipids to a matrix of triglyceride, the erosion of the matrix may be accelerated in vivo.

These surface-active compounds may be used in a content within a range of up to 90 (w/w), and in particular 0.5 to 50 (w/w), with reference to the lipid matrix.

Thus the degradability of the carrier system according to the invention in vivo may be altered not only via the composition of the lipid matrix or of the carrier system, but also with the use of suitable additives.

It will be understood that other additives, such as those known for use in release-controlling systems, may be added if required.

Such additives may be, inter alia, other problematic medicaments, stabilising compounds such as electrolytes and buffering substances.

According to another embodiment, the active substance itself may act as a release-controlling substance, thereby completely or partially replacing the latter within the carrier system. In this case, the active-substance content may be 1 wt. % or more, in particular 2 wt. % or more, with reference to the carrier system.

Examples of active substances which are suitable for this purpose are insulin, growth factors of the BMP family such as BMP-2, growth factor IGF and erythropoietin (EPO).

The carrier system according to the invention may be available for administration in any geometric shape. They are usually round, oval or cylindrical.

The carrier system may be a multi-layer system, in which a hollow or solid core is surrounded by further layers. The individual layers may be charged with the same or with different active substances; individual layers may also be active substance-free. The active substance may be present in the individual layers in the same or in differing amounts.

In this respect the carrier system may be designed in accordance with requirements and the desired dosage form.

Depending on the dosage form, the carrier system according to the invention may be solid to semi-solid. For example, the carrier system for injection may be used in a semi-solid form.

For use in humans or animals, the carrier system should not melt. The melting point of the carrier system according to the invention is therefore preferably above 40° C., especially 60° C. or more.

The carrier system may be manufactured and charged using the methods for this purpose which are known per se. For example, it may be manufactured by extruding the active substance and the material for the carrier system.

The carrier system is advantageously manufactured and charged by first of all melting the lipid or lipid mixture. The active substance in the solid or undilute form is then stirred into the melt, forming a suspension. If applicable, the release-controlling substance may be mixed in at the same time. The suspension obtained can then be worked up into the desired form.

If required, an organic solvent may be added to the lipid mixture to improve the homogenisation of the lipids; the organic solvent is removed e.g. by evaporation prior to addition of the active substance.

In contrast with known methods, in which the active substance is first of all dissolved in a solvent, according to the invention the carrier system can therefore be charged with active substance without the active substance coming into contact with a solvent. This is especially advantageous in the case of active substances which exhibit low stability in contact with a solvent such as water, etc.

A solid active substance may be added in the form of particles of suitable granule size, which varies according to requirements. The mean granule size is usually in general less than 1000 μm.

Especially advantageous is the addition of the active substance in a micronised form, with a mean granule size of <10 μm, especially <5 μm right into the nanometre range.

Similarly the release-controlling substance may be added in the form of particles of suitable size.

The working up of the active substance and of the release-controlling substance to form particles such as microparticles of the required size may be performed by methods conventionally used for this purpose. For example, the comminution may be performed by milling in suitable mills.

The carrier system according to the invention may advantageously be used to produce pharmaceutical compositions.

Depending on the choice of active substance or pharmaceutical substance employed, the pharmaceutical composition may be used to release the active substance or pharmaceutical substance in vitro or in vivo in living organisms such as humans or animals such as the horse, dog, rabbit, cow, mouse, rat, etc.

Due to the stabilisation of the active substance which, according to the invention, is also possible over a long period, even in regions where medical facilities are poor, living organisms may be treated by means of a single administration of the pharmaceutical composition by a doctor and supplied with necessary problematic medicaments over a lengthy period without further involvement of the doctor. If, for example, a plurality of administrations separated by time intervals are necessary, the release may be controlled accordingly by the provision of correspondingly active substance-free portions in the pharmaceutical composition alongside portions which contain active substance.

The function and mode of action of the carrier system according to the invention will be made especially clear on the basis of the embodiments shown in the drawings.

The figures are as follows:

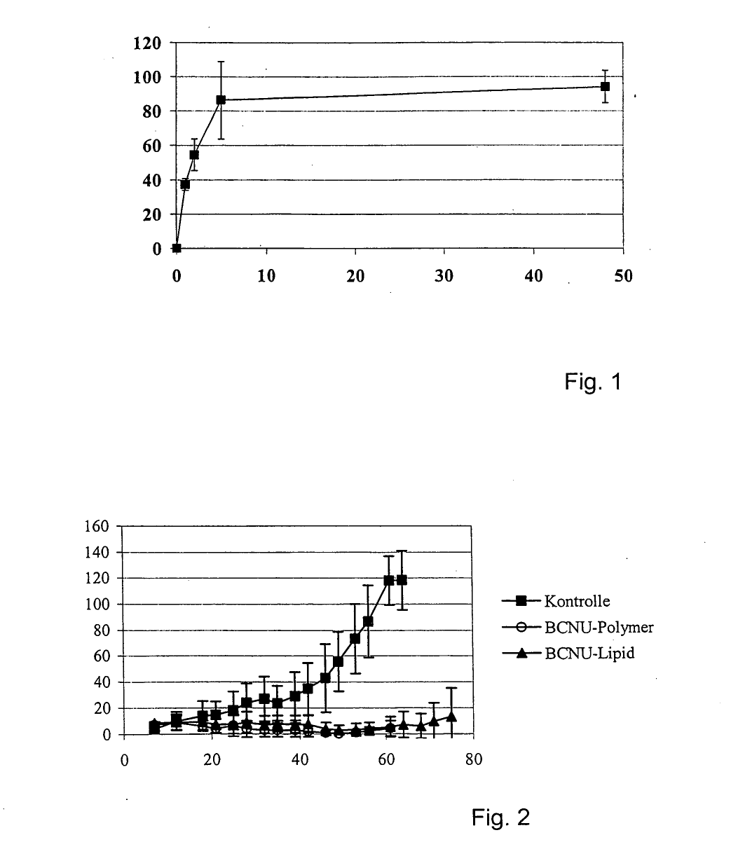

FIG. 1: A diagram showing the stabilising action of the lipids used according to the invention in respect of proteins;

FIG. 2: A diagram comparing the efficacy of BCNU in tumour-bearing nude mice for lipid matrices according to the invention and in conventional polyanhydride matrices;

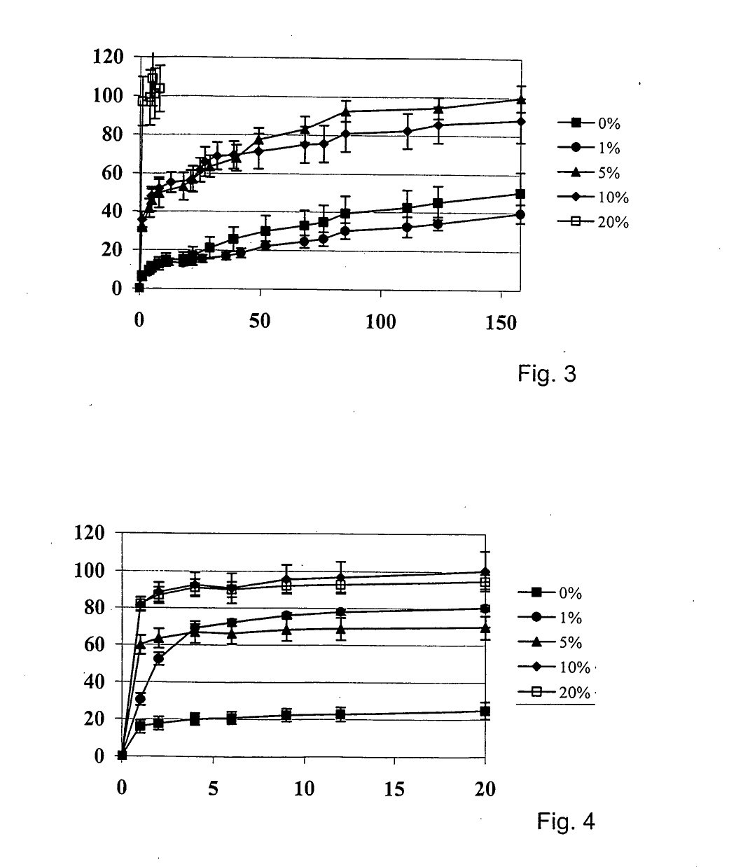

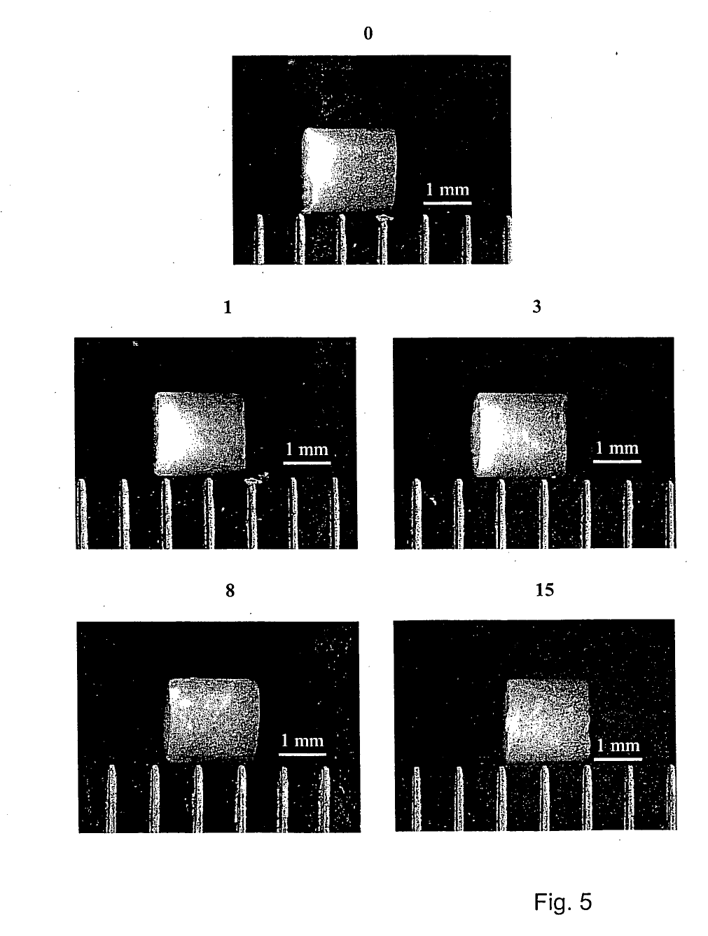

FIGS. 3 and 4: Diagrams showing modification of the rate of release in the carrier system according to the invention as a function of the content of release-controlling substance, and

FIG. 5: Images of a carrier system according to the invention obtained in vivo over a period of 15 days.

FIG. 1 is a diagram in which the activity of the enzyme hyaluronidase in a conventional carrier system composed of poly(1,3-bis[carboxyphenoxypropane]-sebacic acid is shown to be 20:80 of the activity of this enzyme in the matrix composed of glyceryl tripalmitate as the lipid. Whereas the release of the enzyme from the conventional system leads to inactivation, in the case of in-vivo release from the glyceryl tripalmitate matrix 100% of the activity is achieved. This shows that proteins are in principle stabilised by lipids such as those used according to the invention for the carrier system, and also retain their activity over a longer period.

The ordinate gives the release quantity of hyaluronidase in percent, and the abscissa the time in hours.

Apart from stabilisation of problematic active substances, the carrier systems according to the invention are also characterised by better tolerability in a biological system by comparison with numerous polymers such as are conventionally used for carrier systems. In this regard, reference is made to the deposition of monomers, the degradation products of polymer-based carrier systems, and oedematisation following the application within the central nervous system (CNS) of polymeric implants containing cytostatics. Problems of this type are caused, inter alia, by the use of polyanhydrides, for example poly(1,3-bis[carboxyphenoxypropane]-co-sebacic acid) in a ratio of 20:80.

Polyanhydrides are used within the CNS mainly in the treatment of glioblastoma multiforme, for protecting the hydrolysis-sensitive active substance BCNU from inactivation due to water. However, the use of polyanhydrides can lead to an accumulation of monomers within the CNS which persists for months and can lead to local irritation and even oedema.

According to the invention it has been found that with the use of lipids, as with conventional polyanhydrides, the diffusion of water into a matrix can be slowed and the stability of the active substance in vivo guaranteed. Thus the effectiveness of BCNU in tumour-bearing nude mice (U78 MG tumour subcutaneously inoculated) is the same for a lipid matrix according to the invention and for a conventional polyanhydride matrix, as shown in FIG. 2.

In FIG. 2, the area of the tumour is plotted in nm² on the ordinate, and the time in days on the abscissa.

This shows that, in addition to the already described stabilisation of problematic medicaments, the harmful effects of polymers on tissue can also be prevented. Lipids such as, for example, cholesterol are a natural constituent of tissues in the CNS, in contrast with the conventionally used polymers such as the polyanhydrides.

Other materials from the lipid group may be converted or broken down naturally within the biological system in the metabolism, without influencing physicochemical parameters such as the pH or the osmotic pressure.

On the basis of the above-mentioned knowledge concerning the efficacy of the lipids used according to the invention, it is for example possible to replace polyanhydrides in implants, such as those described for example in U.S. Pat. No. 6,086,908, or in microparticles or implants for use within the CNS.

FIGS. 3 and 4 show diagrams illustrating the dependence of the rate of release on the proportion of release-controlling substance in the carrier system according to the invention. In the embodiment shown in FIGS. 3 and 4, a lipid matrix composed of glycerol tripalmitate with differing gelatin contents is used as the release-controlling substance. The carrier system used is cylindrical in shape. FIG. 3 shows the controlled release of pyranine, a low-molecular fluorescent pigment, over a period of several weeks, and FIG. 4 the release of bovine serum albumin fluorescence-labelled with tetramethylrhodamine (TAMRA-BSA). In both cases release of the active substance over a number of weeks or months is possible.

The proportion of gelatin within the carrier system is 0, 1, 5, 10 and 20%.

The quantity released in percent is shown on the ordinate, and the time in days on the abscissa.

The release which is possible in vivo according to the invention even over long periods from the carrier systems according to the invention is also ensured in that the carrier systems per se are similarly stable over long periods in vivo.

Thus experiments with nude mice demonstrate that, following subcutaneous implantation of carrier systems based on glyceryl trimyristate, no significant change in the geometry of the carrier system is detectable even after 15 days (FIG. 5). The images in FIG. 5 show the carrier system after 0, 1, 3, 8 and 15 days, the numbers above the images representing the number of days.

As shown above, by combining lipids having differing physical properties, solid carrier systems may be obtained and worked up to form implants or microparticles, or semi-solid systems may also be produced, which are especially suitable for administration e.g. with the aid of a needle. The examples given below are intended to further illustrate the present invention.

### EXAMPLE 1

Production of an insulin-charged carrier system

To produce insulin-charged lipid microparticles based on glyceryl tripalmitate, the proteins were dispersed as a solid substance in a melt of the lipid.

To this end, the lipid was heated to at least 5° C. above the melting point (approximately 70° C.). The insulin present as the solid substance was dispersed in the melt with the use of an Ultraturrax at approximately 10,000 rpm for one minute. The dispersion obtained was then sprayed with a single-substance nozzle. The particles which formed solidified due to cooling in air.

To influence the release of the active substance by means of a release-controlling substance, the active substance and the release-controlling substance were first of all worked up to form an aqueous solution. When using gelatin as the release-controlling substance, an approximately 5% gel (m/m) was first of all produced by dissolving the active substance. The gel was then freeze-dried. The resulting solid substance was thereafter dispersed in a lipid melt as described above, and worked up to form particles.

### EXAMPLE 2

Substances used for the release from the carrier system (for example pyranine or tetramethylrhodamine-labelled bovine serum albumin (TAMRA-BSA) were dispersed in the desired quantity in an aqueous 15% (m/m) gelatin solution. 100-μl portions of this dispersion were pipetted into each opening in a 96-hole plate, and freeze-dried. The lyophilisate was comminuted in a mortar, and mixed with glyceryl trimyristate in a desired percentage. Powder mixtures with a content of up to 30% (m/m) of gelatin resulted. Cylindrical carrier systems were produced by compressing the corresponding proportions by weight of gelatin with lipid granulate. To this end, 7 mg of substance was produced in a 2-mm pressing tool using a compressive force of 250 N over 10 sec. In this way, carrier system charged with pyranine or fluorescence-labelled bovine serum albumin (TAMRA-BSA) were produced in the composition, as shown in Table 1.

TABLE 1
Composition of carrier systems charged with
pyranine or TAMRA-BSA

| | | Glyceryl trimyristate |
|---|---|---|
| | Gelatin [mg] | [mg] |
| Pyranine [mg] | | |
| 6 | — | 194 |
| 6 | 2 | 192 |
| 6 | 10 | 184 |
| 6 | 20 | 174 |
| 6 | 40 | 154 |
| TAMRA-BSA [mg] | | |
| 2 | — | 98 |
| 2 | 1 | 97 |
| 2 | 5 | 93 |
| 2 | 10 | 88 |
| 2 | 20 | 78 |

### EXAMPLE 3

To test the release of model substances from carrier systems depending on the content of release-controlling substance, the cylinders obtained in Example 1 were incubated at 37° C. in 40 ml of phosphate buffer at pH 7.4. The release of the substances was monitored by measuring the fluorescence intensity. To this end, pyranine was excited at 407 nm and the emission measured over 436 nm. The TAMRA-BSA content was determined at a wavelength of 572 nm with excitation at 541 nm. FIG. 3 shows the release of pyranine (mean values for n=5), FIG. 4 the release of TAMRA-BSA (mean values for n=4). The two figures confirmed that the release of the substances is optimally controlled via the gelatin content.

### EXAMPLE 4

To test the stabilising properties of the carrier systems, glyceryl trimyristate was worked up with hyaluronidase. To this end, 25 mg neopermease, a mixture of 200,000 IU hyaluronidase and gelatin, was mixed with 325 mg of lipid and compressed as described in Example 1 to form cylindrical carrier systems weighing 7 mg. The gelatin content of this mixture was approximately 7.1%. To determine the release of the enzyme, 5 matrices were incubated with phosphate buffer as described in Example 2, and the release determined by measuring the activity via the Morgan-Elson reaction (Muckenschnabel I., Cancer Lett. 131(1) (1998) 13 to 20). FIG. 1 shows that all the activity is liberated; thus the enzyme was stabilised in the carrier system.

1. A carrier system for the stabilization and controlled release of active substances, the carrier system comprising:
at least one lipid matrix having a solubility in water of 1 part of lipid matrix in at least 30 parts or more of water; and
at least one release-controlling substance which is insoluble in the lipid matrix.

2. The carrier system according to of claim 1,
wherein the carrier system does not melt in biological environments.

3. The carrier system of claim 1,
wherein the solubility of the release-controlling substance is 1 part of substance to at least 10,000 parts of lipid matrix.

4-19. (canceled)

20. The carrier system of claim 2, wherein the solubility of the release-controlling substance is 1 part of substance to at least 10,000 parts of lipid matrix.

21. The carrier system of claim 1,
wherein the release-controlling substance has a solubility in water of 1 part of substance to 30 parts of water or less than 30 parts of water.

22. The carrier system of claim 2,
wherein the release-controlling substance has a solubility in water of 1 part of substance to 30 parts of water or less than 30 parts of water.

23. The carrier system of claim 3,
wherein the release-controlling substance has a solubility in water of 1 part of substance to 30 parts of water or less than 30 parts of water.

24. The carrier system of claim 20,
wherein the release-controlling substance has a solubility in water of 1 part of substance to 30 parts of water or less than 30 parts of water.

25. The carrier system of claim 1,
wherein the release-controlling substance is selected from among polymers of the class of the hydrogel-forming polymers, the polysaccharides, the proteins and peptides, the polyethers and polyesters, an electrolyte and monosaccharides and disaccharides and amino acids, as well as combinations thereof.

26. The carrier system of claim 2,
wherein the release-controlling substance is selected from among polymers of the class of the hydrogel-forming polymers, the polysaccharides, the proteins and peptides, the polyethers and polyesters, an electrolyte and monosaccharides and disaccharides and amino acids, as well as combinations thereof.

27. The carrier system of claim 3,
wherein the release-controlling substance is selected from among polymers of the class of the hydrogel-forming polymers, the polysaccharides, the proteins and peptides, the polyethers and polyesters, an electrolyte and monosaccharides and disaccharides and amino acids, as well as combinations thereof.

28. The carrier system of claim 20,
wherein the release-controlling substance is selected from among polymers of the class of the hydrogel-forming polymers, the polysaccharides, the proteins and peptides, the polyethers and polyesters, an electrolyte and monosaccharides and disaccharides and amino acids, as well as combinations thereof.

29. The carrier system of claim 21,
wherein the release-controlling substance is selected from among polymers of the class of the hydrogel-forming polymers, the polysaccharides, the proteins and peptides, the polyethers and polyesters, an electrolyte and monosaccharides and disaccharides and amino acids, as well as combinations thereof.

30. The carrier system of claim 22,
wherein the release-controlling substance is selected from among polymers of the class of the hydrogel-forming polymers, the polysaccharides, the proteins and peptides, the polyethers and polyesters, an electrolyte and monosaccharides and disaccharides and amino acids, as well as combinations thereof.

31. The carrier system of claim 23,
wherein the release-controlling substance is selected from among polymers of the class of the hydrogel-forming polymers, the polysaccharides, the proteins and peptides, the polyethers and polyesters, an electrolyte and monosaccharides and disaccharides and amino acids, as well as combinations thereof.

32. The carrier system of claim 24,
wherein the release-controlling substance is selected from among polymers of the class of the hydrogel-forming polymers, the polysaccharides, the proteins and peptides, the polyethers and polyesters, an electrolyte and monosaccharides and disaccharides and amino acids, as well as combinations thereof.

33. The carrier system of claim 25,
wherein the at least one release-controlling substance is selected from the group consisting of polyethylene glycol and a hydrogel-forming polymer.

34. The carrier system of claim 26,
wherein the at least one release-controlling substance is selected from the group consisting of polyethylene glycol and a hydrogel-forming polymer.

35. The carrier system of claim 27,
wherein the at least one release-controlling substance is selected from the group consisting of polyethylene glycol and a hydrogel-forming polymer.

36. The carrier system of claim 28,
wherein the at least one release-controlling substance is selected from the group consisting of polyethylene glycol and a hydrogel-forming polymer.

37. The carrier system of claim 29,
wherein the at least one release-controlling substance is selected from the group consisting of polyethylene glycol and a hydrogel-forming polymer.

38. The carrier system of claim 30,
wherein the at least one release-controlling substance is selected from the group consisting of polyethylene glycol and a hydrogel-forming polymer.

39. The carrier system of claim 31,
wherein the at least one release-controlling substance is selected from the group consisting of polyethylene glycol and a hydrogel-forming polymer.

40. The carrier system of claim 32,
wherein the at least one release-controlling substance is selected from the group consisting of polyethylene glycol and a hydrogel-forming polymer.

41. The carrier system of claim 1, wherein the at least one release-controlling substance is not of animal origin.

42. The carrier system of claim 1, wherein the active substance is present in the carrier system in a content of less than 10 wt. %.

43. The carrier system of claim 1, further comprising at least one active substance.

44. The carrier system of claim 35, wherein the active substance is a problematic active substance.

45. The carrier system of claim 1, wherein the weight increase of the carrier system associated with swelling in water is below 20%.

46. A pharmaceutical composition containing a carrier system, the carrier system comprising:
at least one problematic active substance;
at least one lipid matrix, the lipid matrix having a solubility in water of 1 part of lipid matrix in at least 30 parts or more of water; and
at least one release-controlling substance which is insoluble in the lipid matrix.

47. The pharmaceutical composition according to claim 46, wherein the problematic active substance is a medicament based on proteins, peptides and antisense oligonucleotides.

48. The pharmaceutical composition of claim 46, wherein the medicament is selected from among a cytokine, growth factor, a cytostatic, insulin, hyaluronidase and erythropoietin.

49. The pharmaceutical composition of claim 47, wherein the medicament is selected from among a cytokine, growth factor, a cytostatic, insulin, hyaluronidase and erythropoietin.

50. The pharmaceutical composition of claim 46, wherein the active substance is D-arginyl-L-arginyl-L-prolylglycyl-3-(2-thienyl)-L-alanyl-L-seryl-(3R)-1,2,3-tetrahydro-3-isoquinolinocarbonyl-(2S,3aS,7aS)-octahydro-1H-indo-1-2-carbonyl-L-arginine.

51. A method of using a carrier system to prepare a pharmaceutical composition, comprising the steps of:
preparing a carrier system comprising:
at least one problematic active substance;
at least one lipid matrix, the lipid matrix having a solubility in water of 1 part of lipid matrix in at least 30 parts or more of water; and
at least one release-controlling substance which is insoluble in the lipid matrix; and
using the carrier system to prepare the pharmaceutical composition.

52. The method of claim 51, wherein the pharmaceutical composition is for the parenteral administration of active substances.

53. The method of claim 51, wherein the pharmaceutical composition is for the administration of an active substance by injection.

54. The method of claim 51, wherein the pharmaceutical composition is for the in-vitro or in-vivo release of problematic active substances.

55. The method of using a carrier system of claim 50, wherein the active substance is a problematic active substance.

56. The method of using a carrier system of claim 51, wherein the active substance is a problematic active substance.

57. The method of using a carrier system of claim 52, wherein the active substance is a problematic active substance.

58. The method of using a carrier system of claim 53, wherein the active substance is a problematic active substance.

59. The method of using a carrier system of claim 54, wherein the active substance is a problematic active substance.

60. Use of lipids having a water solubility of 1 part of lipid to at least 30 parts or more of water for the stabilisation of problematic active substances.
